(19) United States
(12) Patent Application Publication
(10) Pub. No.: US 2006/0216716 A1
(43) Pub. Date: Sep. 28, 2006

(54) Immunologic activity measures for t helper on-associated conditions

(76) Inventors: Lawrence J. Saubermann, Dover, MA (US); Francis A. Farraye, Medfield, MA (US); Victor M. Morales, Hollis, NH (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/568,364

(22) PCT Filed: Aug. 10, 2004
(86) PCT No.: PCT/US04/26236

§ 371 Date: Feb. 14, 2006

Related U.S. Application Data
(60) Provisional application No. 60/495,035, filed on Aug. 14, 2003.

(51) Int. Cl. C12Q 1/68; G01N 33/567

(52) U.S. Cl. 435/6; 435/7.21

(57) ABSTRACT

A method of providing a reproducible and clinically relevant disease activity measurement for a group of immunologic disorders that share features of a T “helper” 1 (Th1) immune activation phenotype is disclosed. Th1 responses are characterized by the release of a set of pro-inflammatory substances by white blood cells (WBC) By evaluating the ex vivo activation response of whole blood in terms of the extent of release of Th1 produced pro-inflammatory substances in blood (i.e., WBC activity) following activation with a Th1-associated stimulant, the level of, e.g., disease activity or immunosuppression in a specific individual can be determined. The method of the invention provides a disease activity marker for a wide variety of Th1 inflammatory responses, including Crohn's Disease, psoriasis, rheumatoid arthritis, Systemic Lupus Erythematosus (SLE), multiple sclerosis and solid organ transplant rejection.

### CROSS REFERENCE TO RELATED APPLICATIONS

This application claims priority from U.S. Provisional Application No. 60/945,035, filed Aug. 14, 2003, the whole of which is incorporated by reference herein.

### STATEMENT REGARDING FEDERALLY SPONSORED RESEARCH OR DEVELOPMENT

None

### BACKGROUND OF THE INVENTION

Non-specific inflammation associated blood tests, such as the erythrocyte sedimentation rate (ESR) or c-reactive protein (CRP) levels, are currently available. These tests are generally associated with both acute and chronic inflammation states. However, the majority of the time, such tests are not clinically useful as they cannot be used as an accurate measure of the level of immunologic disease activity and, therefore, do not generally result in changes in disease management. Other methods for assessing disease activity specifically in subjects with Th1-associated diseases include complicated formulas that can combine history, physical examination results, laboratory, radiologic, endoscopic and other diagnostic findings to produce a clinical disease activity index score. These scoring systems are primarily used in comparing clinical efficacy between therapies in research studies rather than being used in routine clinical practice. It would be desirable to have clinically suitable inflammation-associated blood tests that could accurately reflect the level of immune activation or suppression, thereby providing a measure for repeated comparisons to assess therapies and predict disease relapses.

### BRIEF SUMMARY OF THE INVENTION

The invention is directed to providing a reproducible and clinically relevant disease activity measurement for a group of immunologic disorders that share features of a T “helper” 1 (Th1) immune activation phenotype. Th1 responses are characterized by the release of a set of pro-inflammatory substances by white blood cells (WBC), which include cytokines, e.g., IL-12, Tumor Necrosis Factor-alpha, and Interferon-gamma; and also include chemokines (chemotactic cytokines), e.g., CXCL9 (MIG), CXCL10 (IP-10, IP10), and CXCL11 (ITAC, I-TAC). By evaluating the ex vivo activation response of whole blood in terms of the extent of release of Th1 produced pro-inflammatory substances in blood (i.e., WBC activity) following activation with a Th1-associated stimulant, a climician can assess the level of, e.g., disease activity or immunosuppression in a specific individual. The method of the invention provides a disease activity marker for a wide variety of Th1 inflammatory responses, e.g., for Crohn's Disease, psoriasis, rheumatoid arthritis, Systemic Lupus Erythematosus (SLE), multiple sclerosis or solid organ transplant rejection. This method can be used, e.g., to serially monitor levels of immune system activation and its suppression for those individuals on chronic immunosuppressants or immunomodulator therapies, to evaluate a response to immunosuppressive therapy, or to predict a relapse or flare in disease activity levels. The method of the invention can incorporate the use of existing technologies, including the use of commercially available cytokines, or other forms of white blood cell activators, to perform cytokine/chemokine measurements on blood samples using, e.g., standardized antibody based detection kits.

Thus, the invention is generally directed to an ex vivo method of measuring, for an individual, the individual's level of Th1 immune activity and the adequacy of the individual's immunosuppressive levels for prevention of an exacerbation, or to evaluate the immunosuppressive treatment, of a Th1-associated condition. The method of the invention includes the steps of providing an individual with an appropriate Th1-associated condition, collecting a blood sample that includes the WBC from the individual, adding a pro-inflammatory stimulant to the blood sample, incubating the blood sample with the stimulant and assaying the stimulated sample for the extent of release of a pro-inflammatory substance from the WBC, the extent of release of the pro-inflammatory substance in response to the pro-inflammatory stimulant being indicative of the level of immunosuppression in the individual.

The advantages of having a Th1 disease activity marker measurement method, as outlined herein, are in the ability of the marker to accurately reflect the level of immune activation in the tested individual and to provide a measure for repeated comparisons to use in assessing therapies and predicting impending disease relapses. The method according to the invention can be performed quickly using existing technologies and a single blood sample.

### BRIEF DESCRIPTION OF THE SEVERAL VIEWS OF THE DRAWINGS

Other features and advantages of the invention will be apparent from the following description of the preferred embodiments thereof and from the claims, taken in conjunction with the accompanying drawings, in which:

FIG. 1 is a scatter plot showing representative examples of the chemokine CXCL10 response by WBC following interferon-gamma stimulation in subjects with either Crohn's Disease or Ulcerative Colitis;

FIG. 2a is a graph showing a comparison of Crohn's Disease subjects' levels of overall clinical activity, as determined by a gastroenterologist in a blind test, to the level of interferon-gamma-activated CXCL10 in the blood at 3 hours;

FIG. 2b is a representation of the % change in CXCL10 levels from the clinically active to the clinically less active disease state, as shown in FIG. 2a, for each subject;

FIG. 3 is a graph showing a longitudinal assessment of CXCL10 levels for Crohn's Disease subjects reported as clinically inactive by a gastroenterologist in a blind test;

FIG. 4 is a table presenting an analysis of the effects of the individual medications on the activated CXCL10 levels over time within Crohn's Disease subjects;

FIG. 5 is a graph demonstrating activated CXCL9 levels following interferon-gamma stimulation over time (between two sampling periods) in comparison to a blind assessment of overall clinical disease activity for a small number of Crohn's Disease subjects;

FIG. 6 is a scatter plot showing the effects of different forms of activation and a combination of activating cytokines on CXCL9 levels in Crohn's Disease subjects;

FIG. 7 is a scatter plot showing a comparison of the effect of Crohn's Disease subject medication usage on activated CXCL9 levels;

FIG. 8 is a scatter plot showing a comparison of Ulcerative Colitis subject CXCL9 levels following different forms of activation; and

FIG. 9 is a scatter plot showing IP-10 (CXCL10) levels after activation in multiple Th1-associated disease states and the association with disease activity. (Key: CD=Crohn's Disease, UC=Ulcerative Colitis, RA=Rheumatoid Arthritis, VS=Vasculitis, SLE=Systemic Lupus Erythematosus, Normal=normal volunteers).

### DETAILED DESCRIPTION OF THE INVENTION

In one embodiment of the method of the invention, an antibody derived serologic measurement of one or more Th1-associated cytokines, e.g., IL-12, tumor necrosis factor-alpha, and interferon-gamma, or chemokines (chemotactic cytokines), e.g., CXCL9 (MIG), CXCL10 (IP-10, IP10), and CXCL11 (ITAC, I-TAC), or any other inducible protein, after ex vivo activation by a cytokine or other stimulant, such as interferon-gamma, is determined. Stimulation can also be provided by a combination of cytokines, such as interferon-gamma and tumor necrosis factor-alpha. This embodiment of the assay system according to the invention is particularly appropriate for measuring the level of immune activation in the Th1 inflammatory bowel disease, Crohn's Disease.

Following informed consent, a heparinized whole blood sample. (e.g., 1-5 cc) is collected from Th1—disease subjects (e.g., Crohn's Disease) with either an active or inactive disease state, as defined clinically. A pro-inflammatory stimulant, e.g., interferon-gamma, is added at a concentration of approximately 1000 units/cc and incubated at 37° C. A similar control sample without the pro-inflammatory stimulant is also incubated. At an appropriate time interval, e.g., 3 hours, the samples are centrifuged to allow collection of the plasma, or serum (the non-cell containing portion of the blood). An aliquot, e.g., 5-20 μL, of the plasma/serum from each sample is added to one well of, e.g., a 96 well ELISA (Enzyme-Linked Immunosorbent Assay) plate that has been previously coated with a capture antibody for a proinflammatory substance associated with the stimulant used, e.g., the interferon-gamma inducible and Th1-related chemokine CXCL10 (−IP10, IP-10) protein. In a standard sandwich ELISA detection, the plasma/serum is allowed to incubate with a capture antibody. Then, the sample is removed, the plate is washed multiple times and an antibody labeled with an enzyme is applied (detection antibody). Finally, and the plate is again washed repeatedly. Depending on the enzyme used for the detection antibody, a color change becomes evident over time after addition of the proper enzyme substrate, which can then be detected using specified light wavelength measurements. The light absorbance can be measured using a standard ELISA plate reader, which in turn produces a number. By comparing this number to a standard curve for CXCL10, a quantified amount of CXCL10 can be determined, or alternatively, the absorbance number itself may be used. The clinician receives a value for the inducible chemokine, which can then be compared to prior values for the patient, or with other values for the specific Th1 disease, in order to determine the level of immunosuppression or disease activity in the tested individual.

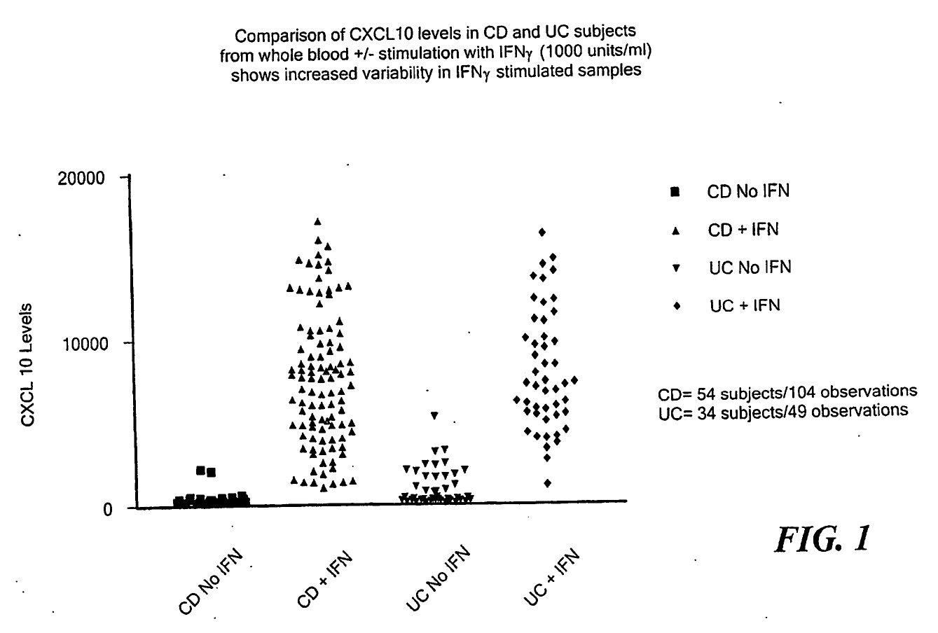

FIG. 1 shows representative examples of the chemokine response by a subject's WBCs following cytokine stimulation, as practiced in the method of the invention. Referring to FIG. 1, it can be seen that activation/stimulation with interferon-gamma results in a larger spread and an increase in the measurable values for the chemokine CXCL10 at 3 hours post stimulation in inflammatory bowel subjects with Crohn's Disease or Ulcerative Colitis. This response also occurs in normal individuals as well.

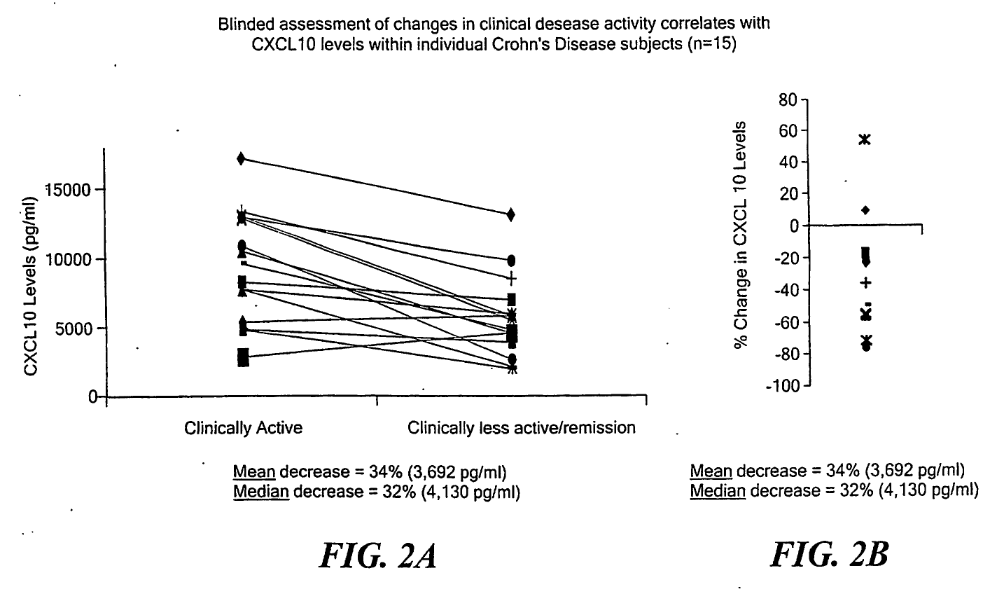

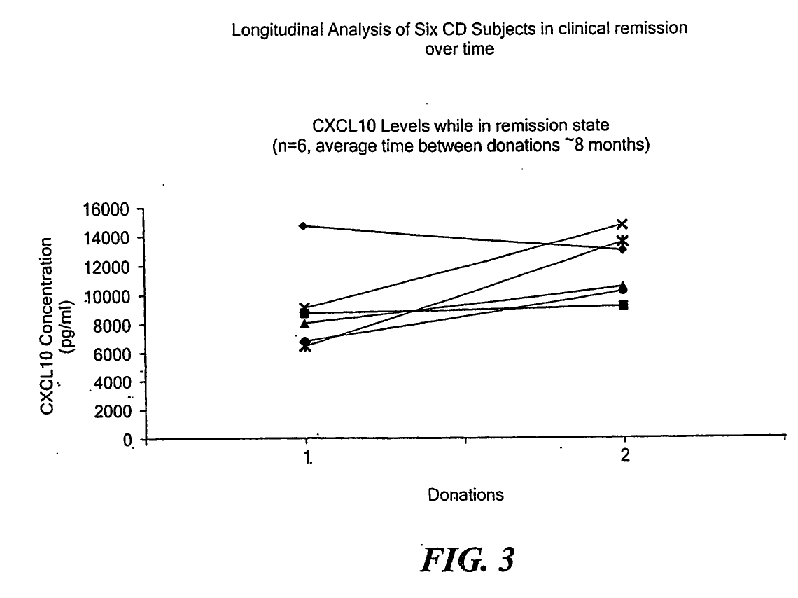

A blind assessment of changes in clinical disease activity shows a correlation with changes in CXCL10 levels in individual Crohn's Disease subjects. FIG. 2a is a comparison of the levels of overall clinical activity in Crohn's Disease subjects, as determined by a gastroenterologist in a blind test, to the level of interferon-gamma-activated CXCL10 in the blood at 3 hours. This change in level of clinical activity is represented as a % change in FIG. 2b, with the mean decrease being 34% and the median 32%. A longitudinal analysis of six Crohn's Disease subjects in clinical remission is reported in FIG. 3. The average time between the subjects' blood donations was approximately eight months. This graph generally notes a trend towards gradually increasing levels or steady levels of the chemokine CXCL10 in these subjects over time.

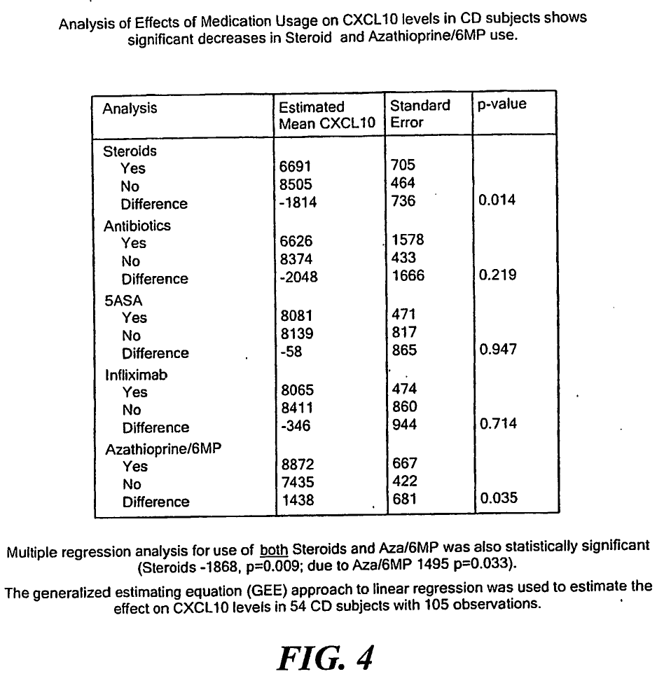

Medication usage can have a statistically significant effect on stimulated chemokine levels in Crohn's Disease subjects. The Table shown in FIG. 4 presents an analysis of the effects of individual medications on activated CXCL10 levels over time within individual Crohn's Disease subjects. As shown, both steroid and azathioprine/6MP usage brought about significant changes in activated CXCL10 levels over time. The effect of simultaneous treatment these two medications was also significant.

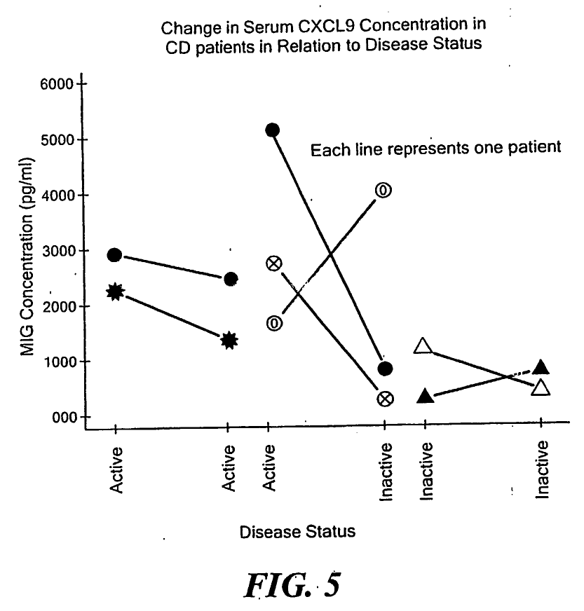

In other experiments, changes in serum concentration of another chemokine, CXCL9, was also followed in Crohn's Disease subjects. As shown in FIG. 5, the levels of activated CXCL9 (MIG) levels following interferon-gamma stimulation over time (between two samples) is correlated with disease status. For patients whose disease status did not change, the levels of CXCL9 remained relatively flat. In contrast, for the three patients whose disease status was observed to go into remission, in a blind assessment of overall clinical disease activity, the percent change in CXCL9 levels was dramatic.

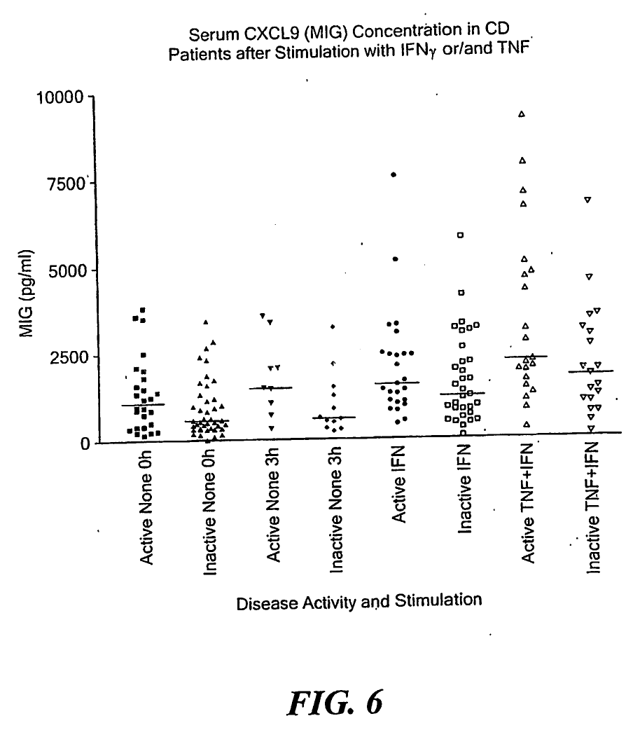

The effects of different forms of activation and a combination of activating cytokines on CXCL9 levels in Crohn's Disease subjects was also studied. As can be seen in FIG. 6, serum CXCL9 concentration levels and the spread of CXCL9 values increased after stimulation with interferon-gamma or after a combination of interferon-gamma and tumor necrosis factor-alpha.

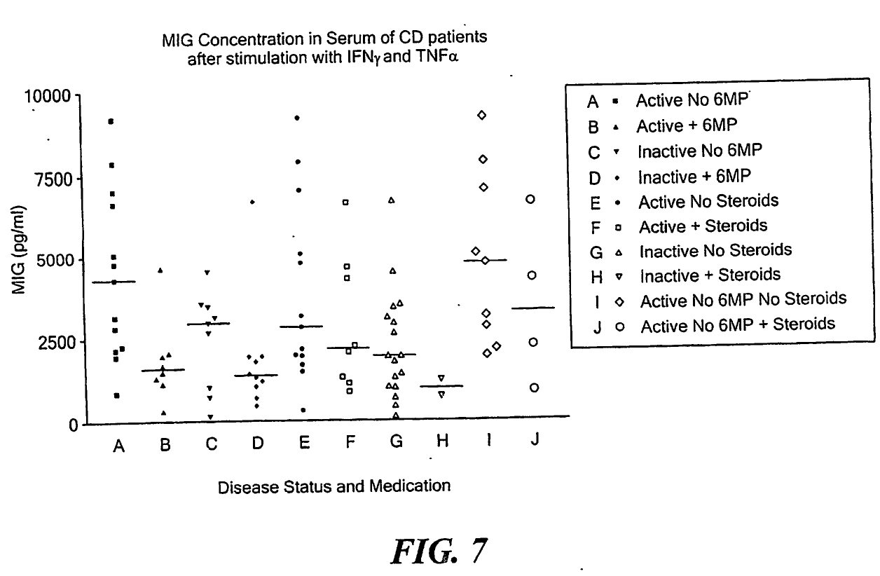

In a subsequent experiment, a combination of stimulating cytokines (interferon-gamma and tumor necrosis factor-alpha) was administered to Crohn's Disease patients and the effects of medication studied. FIG. 7 shows a comparison of Crohn's Disease subject medication usage on activated CXCL9 levels. As can be seen, the use of 6 MP, steroids or a combination thereof all brought about a change in CXCL9 levels in Crohn's Disease subjects, whether the patient's disease status was active or in remission.

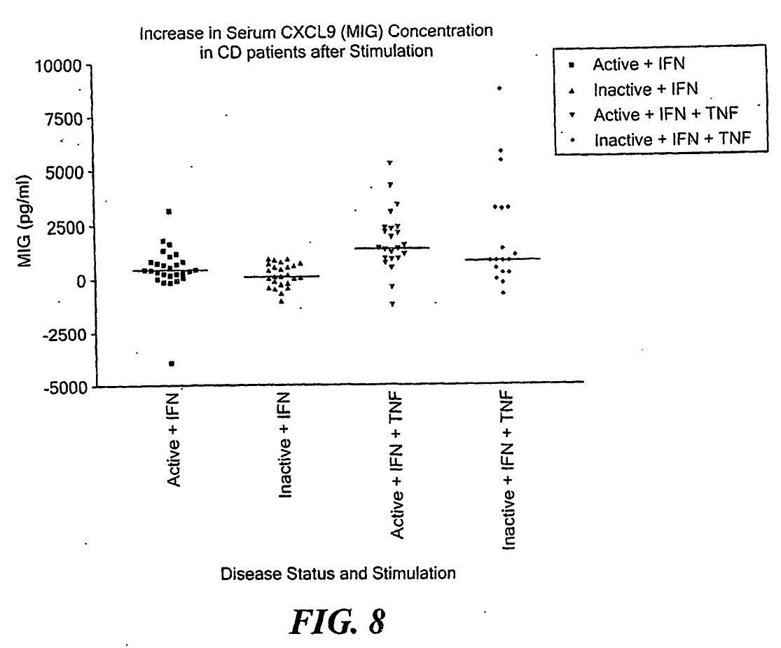

FIG. 8 shows that different forms of activation and a combination of activating cytokines also affects CXCL9 levels in subjects having Ulcerative Colitis. As can be seen in FIG. 8, serum CXCL9 concentration levels and the spread of CXCL9 values increased after stimulation with interferon-gamma or after a combination of interferon-gamma and tumor necrosis factor-alpha for subjects having both active and inactive forms of the disease.

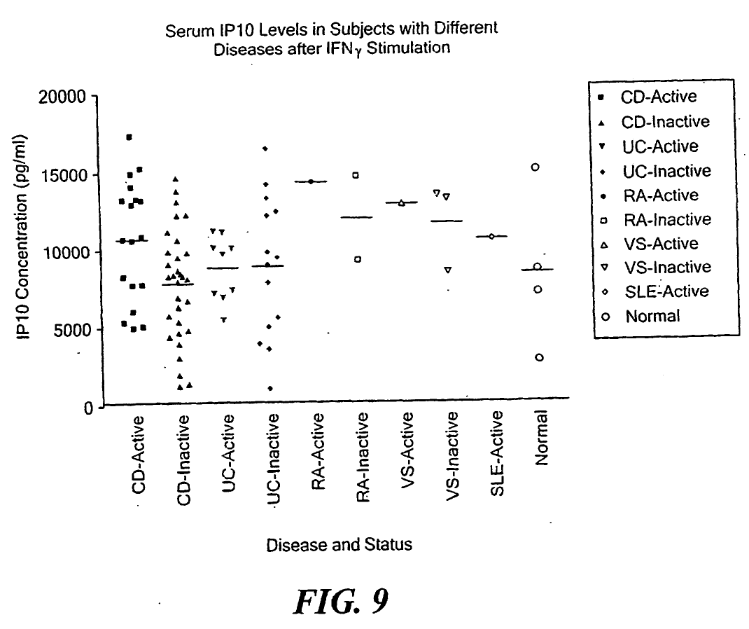

Subjects having a variety of different diseases also show elevated CXCL10 levels following activation. FIG. 9 shows the increase in CXCL10 levels in multiple Th1-associated disease states and the corresponding association with disease activity. (Key: CD=Crohn's Disease, UC=Ulcerative Colitis, RA=Rheumatoid Arthritis, VS=Vasculitis, SLE=Systemic Lupus Erythematosus, Normal=normal volunteers.)

Other conditions that can be monitored using the method of the invention include:
1. Inflammatory conditions of the bowel, including but not limited to Crohn's disease, Celiac Sprue (gluten sensitive enteropathy), ulcerative colitis, Clostridium difficile infection (antibiotic-associated diarrhea), or any other enteric acute or chronic infections that have Th1 cytokine production associated with their clinical manifestation.
2. Inflammatory conditions of the liver, including but not limited to acute and chronic viral hepatitis B or C, autoimmune hepatitis, primary sclerosing cholangitis and primary biliary cirrhosis.
3. Acute and chronic infections, including but not limited to bacterial, viral, or parasitic diseases, such as tuberculosis, giardiasis, cytomegalovirus and HIV.
4. Inflammatory conditions of the eyes, including but not limited to uveitis and scleritis.
5. Inflammatory conditions of the lungs, including but not limited to asthma, idiopathic pulmonary fibrosis, chronic obstructive lung disease and hypersensitivity pneumonitis.
6. Inflammatory conditions of the skin, including but not limited to psoriasis, atopic dermatitis and eczema.
7. Inflammatory conditions of the neurologic system, including but not limited to multiple sclerosis and Guillaume-Barre syndrome.
8. Inflammatory conditions of the musculoskeletal system, including but not limited to arthritis, especially rheumatoid arthritis (juvenile and adult), spondlyarthropathies (ankylosing spondylitis) and myositis.
9. Inflammatory conditions of the renal system, including but not limited to glomerulonephritis.
10. Inflammatory conditions of the pancreas and endocrinologic systems, including but not limited to type I and type II diabetes mellitus and thyroiditis.
11. Inflammatory conditions of the cardiovascular system, including but not limited to myocarditis, vascultitis or athersclerosis.
12. Systemic immune or autoimmune diseases including but not limited to lupus erythromatosus, scleroderma, sarcoidosis and amyloidosis.
13. Allograft organ transplant inflammation or rejection, including but not limited to renal, cardiac, small bowel, pancreatic and liver transplants. Also including graft vs. host disease in the setting of allograft bone marrow transplant.
14. Conditions associated with, or in the monitoring of the treatment of, primary or metastatic cancers, including but not limited to cancers of the skin cutaneous T cell lymphomas, B cell lymphomas and melanoma.
15. Inflammatory conditions involving animals, including but not limited to diabetes and inflammatory bowel disease.

### OTHER EMBODIMENTS

Variations and modifications include the potential to use alternative Th1-related cytokines or other stimulants, e.g., tumor necrosis factor-alpha, the interleukins or bacterial-associated products, such as lipopolysaccharide (LPS), lipotechoic acid (LTA) or peptidoglycan (PGN), rather than, or in combination with, interferon-gamma. Clinical testing can be performed for each disease to determine the best stimulant and its dosage, and the responses may vary depending on the form of Th1 disease state. Any compound produced by the WBC in response to Th1-related stimulation and measurable in serum or plasma, e.g., any cytokine or chemokine (chemotactic cytokine), could be used as a marker for an appropriate Th1-related disease state.

The most appropriate cytokine(s) or chemokine(s), e.g., interleukin-12 or CXCL9, CXCL11, as the sole or as additional markers for a specific condition, can be determined using the method described herein. These determinations can be performed quickly due to the availability of large amounts of plasma/serum from a given blood sample.

It is believed that the individual's own activity level over time is more important and more appropriately tied to disease activity than trying to set a particular overall threshold level for a particular disease. In other words, the magnitude of the change, whether positive or negative, in a stimulated chemokine level, may be more important than the actual number. For example, a change from 5000 to 3000 pg/ml in CXCL10 after interferon-gamma activation may be as relevant to one person, as a change from 10000 to 6000 pg/ml in another.

Other methods that could be used to measure chemokine or cytokine levels in the activated blood sample include: 1) assessment of chemokine synthesis by measuring the messenger RNA (mRNA) level for a particular chemokine, or set of chemokines, using PCR, including “real-time” or quantitative PCR methodology; 2) use of other emerging techniques, such as proteomics, or protein “chip” assays, which could quantify the chemokine levels in the blood; 3) measuring intracellular cytokine production by cells using flow cytometric analysis (FACS analysis); 4) identifying chemokine levels after activation using antibody coated beads or any other form of binding and analysis; and 5) measuring metabolism or any residual portions of the chemokines after their production 6) measuring the effect of the chemokine(s) on cultured cells and examining a response from said cells.

Uses

The clinical usefulness of this technology is in providing an immunologic disease activity measure of a Th1-associated condition, e.g., in order to determine an individual's immune activity status and guide a treatment plan, e.g. immunosuppression. If a patient is first diagnosed with a chronic Th1-associated disease, the cellular response to stimulation can provide meaningful information about their level of disease activity. Further usefulness will be observed by using the method of the invention as a measure of adequate immunosuppression for a particular Th1-associated condition and as a predictor of an exacerbation in the disease state.

A kit for practicing the method of the invention includes a pro-inflammatory stimulant associated with a T helper 1 (Th1)—associated condition and instructions for carrying out the method of the invention for an individual having, or suspected of having, the condition.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. An ex vivo method of measuring the level of immune activation and immunosuppression in an individual having, or suspected of having, a T helper 1 (Th1)-associated condition, said method comprising the steps of:
providing an individual having, or suspected of having, a Th1-associated condition;
collecting a blood sample including white blood cells (WBC) from said individual;
adding a pro-inflammatory stimulant to said sample;
incubating said sample with said stimulant; and
assaying in said stimulated sample the extent of release of a pro-inflammatory substance from said WBCs, wherein the extent of release of said pro-inflammatory substance in response to said pro-inflammatory stimulant is indicative of the level of immunologic activity and/or immunosuppression in said individual.

2. The method of claim 1, wherein said Th1-associated condition is selected from the group consisting of Crohn's Disease, psoriasis, rheumatoid arthritis, Systemic Lupus Erythematosus (SLE), multiple sclerosis and solid organ transplant rejection.

3. The method of claim 1, wherein said pro-inflammatory stimulant is interferon-gamma, tumor necrosis factor-alpha, an interleukin or a combination thereof.

4. The method of claim 1, wherein said pro-inflammatory substance is a chemotactic cytokine.

5. The method of claim 4, wherein said chemotactic cytokine is selected from the group consisting of CXCL9 (MIG), CXCL10 (IP-10, IP10) and CXCL11 (ITAC, I-TAC).

6. The method of claim 1, wherein said pro-inflammatory stimulant is a bacterial-associated lipid or polysaccharide.

7. The method of claim 6, wherein said pro-inflammatory stimulant is selected from the group consisting of lipopolysaccharide, lipotechoic acid, peptidoglycan and subunits or components thereof.

8. The method of claim 1, wherein the extent of release of said pro-inflammatory substance is assayed by a method selected from the group consisting of antibody derived serologic measurement of said pro-inflammatory substance; PCR methodology measurement of messenger RNA levels for said pro-inflammatory substance; protein chip assay quantification of said pro-inflammatory substance; measurement of intracellular production of said pro-inflammatory substance by cells using flow cytometric analysis; binding and release measurement of said pro-inflammatory substance; and measurement of a metabolic product of said pro-inflammatory substance.

9. The method of claim 1, wherein the extent of release of said pro-inflammatory substance is assayed by antibody derived serologic measurement.

10. A kit for ex vivo measurement of the level of immunosuppression in an individual having, or suspected of having, a T helper 1 (Th1)-associated condition, said kit comprising:
a pro-inflammatory stimulant associated with said T helper 1 (Th1)-associated condition; and
instructions for carrying out the method of claim 1 for an individual having, or suspected of having, said condition.

11. The kit of claim 10, wherein said pro-inflammatory stimulant is interferon-gamma, tumor necrosis factor-alpha, an interleukin or a combination thereof.

12. The kit of claim 10, wherein said pro-inflammatory stimulant is a bacterial-associated lipid or polysaccharide.

13. The method of claim 12, wherein said pro-inflammatory stimulant is selected from the group consisting of lipopolysaccharide, lipotechoic acid, peptidoglycan and subunits or components thereof.

14. The kit of claim 10, wherein said Th1-associated condition is selected from the group consisting of Crohn's Disease, psoriasis, rheumatoid arthritis, Systemic Lupus Erythematosus (SLE), multiple sclerosis and solid organ transplant rejection.
